SEQUENCE LISTING (139 sequences)

SEQ ID NO: 1 - DNA, 64 bp, Artificial sequence
  single-stranded nucleic acid molecule A1
  misc_feature (16)..(25): n is a, c, g, or t
cgatccttggtgatcnnnnnnnnnncgtcagtgcttgtcttcctaagatgtgtataagagacag

SEQ ID NO: 2 - DNA, 19 bp, Artificial sequence
  Single-stranded nucleic acid molecule A2
ctguctcutatacacauct

SEQ ID NO: 3 - DNA, 27 bp, Artificial sequence
  5end of single-stranded nucleic acid molecule B1
aaaaaaaaaatgtgagccaaggagttg

SEQ ID NO: 4 - DNA, 30 bp, Artificial sequence
  single-stranded nucleic acid molecule B2
gatcaccaaggatcgccatagtccatgcta

SEQ ID NO: 5 - DNA, 22 bp, Artificial sequence
  adapter-1A
tctgctgagtcgagaacgtctc

SEQ ID NO: 6 - DNA, 14 bp, Artificial sequence
  adapter-2A
ctcgactcagcaga

SEQ ID NO: 7 - DNA, 17 bp, Artificial sequence
  primer-F
tgtgagccaaggagttg

SEQ ID NO: 8 - DNA, 22 bp, Artificial sequence
  primer-R
gagacgttctcgactcagcaga

SEQ ID NO: 9 - DNA, 19 bp, Artificial sequence
  single-stranded nucleic acid molecule C
ctgtctcttatacacatct

SEQ ID NO: 10 - DNA, 30 bp, Artificial sequence
  primer 2-F
ttgtcttcctaagatgtgtataagagacag

SEQ ID NO: 11 - DNA, 10 bp, Artificial sequence
  Molecular barcode
atcggaccta

SEQ ID NO: 12 - DNA, 10 bp, Artificial sequence
  Molecular barcode
gattccgtcc

SEQ ID NO: 13 - DNA, 10 bp, Artificial sequence
  Molecular barcode
cggcagtaag

SEQ ID NO: 14 - DNA, 10 bp, Artificial sequence
  Molecular barcode
tcaattaggt

SEQ ID NO: 15 - DNA, 10 bp, Artificial sequence
  Molecular barcode
cggatacgaa

SEQ ID NO: 16 - DNA, 10 bp, Artificial sequence
  Molecular barcode
gctcgttacc

SEQ ID NO: 17 - DNA, 10 bp, Artificial sequence
  Molecular barcode
ttatacgttg

SEQ ID NO: 18 - DNA, 10 bp, Artificial sequence
  Molecular barcode
aacgcgacgt

SEQ ID NO: 19 - DNA, 10 bp, Artificial sequence
  Molecular barcode
gctagcagaa

SEQ ID NO: 20 - DNA, 10 bp, Artificial sequence
  Molecular barcode
ctatcttcct

SEQ ID NO: 21 - DNA, 10 bp, Artificial sequence
  Molecular barcode
aagcaagagc

SEQ ID NO: 22 - DNA, 10 bp, Artificial sequence
  Molecular barcode
tgcgtgcttg

SEQ ID NO: 23 - DNA, 10 bp, Artificial sequence
  Molecular barcode
cggattgccg

SEQ ID NO: 24 - DNA, 10 bp, Artificial sequence
  Molecular barcode
gaatcctgat

SEQ ID NO: 25 - DNA, 10 bp, Artificial sequence
  Molecular barcode
tctggaatga

SEQ ID NO: 26 - DNA, 10 bp, Artificial sequence
  Molecular barcode
atccagcatc

SEQ ID NO: 27 - DNA, 10 bp, Artificial sequence
  Molecular barcode
catcactcac

SEQ ID NO: 28 - DNA, 10 bp, Artificial sequence
  Molecular barcode
cagctgactc

SEQ ID NO: 29 - DNA, 10 bp, Artificial sequence
  Molecular barcode
ttcgcagaca

SEQ ID NO: 30 - DNA, 10 bp, Artificial sequence
  Molecular barcode
ttgtaccaat

SEQ ID NO: 31 - DNA, 10 bp, Artificial sequence
  Molecular barcode
accacaatcg

SEQ ID NO: 32 - DNA, 10 bp, Artificial sequence
  Molecular barcode
ggaagtctgt

SEQ ID NO: 33 - DNA, 10 bp, Artificial sequence
  Molecular barcode
agagtgtgga

SEQ ID NO: 34 - DNA, 10 bp, Artificial sequence
  Molecular barcode
gcttgtggtg

SEQ ID NO: 35 - DNA, 10 bp, Artificial sequence
  Molecular barcode
ttgtcctcta

SEQ ID NO: 36 - DNA, 10 bp, Artificial sequence
  Molecular barcode
attcgctagg

SEQ ID NO: 37 - DNA, 10 bp, Artificial sequence
  Molecular barcode
cgatgactac

SEQ ID NO: 38 - DNA, 10 bp, Artificial sequence
  Molecular barcode
acagctcagc

SEQ ID NO: 39 - DNA, 10 bp, Artificial sequence
  Molecular barcode
tatctaggtt

SEQ ID NO: 40 - DNA, 10 bp, Artificial sequence
  Molecular barcode
gagatggcaa

SEQ ID NO: 41 - DNA, 10 bp, Artificial sequence
  Molecular barcode
cgcaagatct

SEQ ID NO: 42 - DNA, 10 bp, Artificial sequence
  Molecular barcode
gccgatagcg

SEQ ID NO: 43 - DNA, 10 bp, Artificial sequence
  Molecular barcode
ccatcgttgc

SEQ ID NO: 44 - DNA, 10 bp, Artificial sequence
  Molecular barcode
tgaacgatta

SEQ ID NO: 45 - DNA, 10 bp, Artificial sequence
  Molecular barcode
tagagcgaac

SEQ ID NO: 46 - DNA, 10 bp, Artificial sequence
  Molecular barcode
atgtgtgaga

SEQ ID NO: 47 - DNA, 10 bp, Artificial sequence
  Molecular barcode
atcctaacag

SEQ ID NO: 48 - DNA, 10 bp, Artificial sequence
  Molecular barcode
cgcgtctgcg

SEQ ID NO: 49 - DNA, 10 bp, Artificial sequence
  Molecular barcode
gatgatcctt

SEQ ID NO: 50 - DNA, 10 bp, Artificial sequence
  Molecular barcode
gctcaacgct

SEQ ID NO: 51 - DNA, 10 bp, Artificial sequence
  Molecular barcode
atgcatctaa

SEQ ID NO: 52 - DNA, 10 bp, Artificial sequence
  Molecular barcode
agctctggac

SEQ ID NO: 53 - DNA, 10 bp, Artificial sequence
  Molecular barcode
ctatcacgtg

SEQ ID NO: 54 - DNA, 10 bp, Artificial sequence
  Molecular barcode
ggactagtgg

SEQ ID NO: 55 - DNA, 10 bp, Artificial sequence
  Molecular barcode
gccaagtcca

SEQ ID NO: 56 - DNA, 10 bp, Artificial sequence
  Molecular barcode
cctgtcaagc

SEQ ID NO: 57 - DNA, 10 bp, Artificial sequence
  Molecular barcode
tagaggtctt

SEQ ID NO: 58 - DNA, 10 bp, Artificial sequence
  Molecular barcode
tatggcaact

SEQ ID NO: 59 - DNA, 10 bp, Artificial sequence
  Molecular barcode
ctgcgtacat

SEQ ID NO: 60 - DNA, 10 bp, Artificial sequence
  Molecular barcode
atctcattaa

SEQ ID NO: 61 - DNA, 10 bp, Artificial sequence
  Molecular barcode
aagtggcgca

SEQ ID NO: 62 - DNA, 10 bp, Artificial sequence
  Molecular barcode
ggccttaatg

SEQ ID NO: 63 - DNA, 10 bp, Artificial sequence
  Molecular barcode
tctgaggcgg

SEQ ID NO: 64 - DNA, 10 bp, Artificial sequence
  Molecular barcode
cgagccgatt

SEQ ID NO: 65 - DNA, 10 bp, Artificial sequence
  Molecular barcode
gataaccggc

SEQ ID NO: 66 - DNA, 10 bp, Artificial sequence
  Molecular barcode
tcaatattcc

SEQ ID NO: 67 - DNA, 10 bp, Artificial sequence
  Molecular barcode
tccgttgaat

SEQ ID NO: 68 - DNA, 10 bp, Artificial sequence
  Molecular barcode
cagtacagtt

SEQ ID NO: 69 - DNA, 10 bp, Artificial sequence
  Molecular barcode
attgaggtac

SEQ ID NO: 70 - DNA, 10 bp, Artificial sequence
  Molecular barcode
attagaagtc

SEQ ID NO: 71 - DNA, 10 bp, Artificial sequence
  Molecular barcode
caacgcttca

SEQ ID NO: 72 - DNA, 10 bp, Artificial sequence
  Molecular barcode
ggatcgcacg

SEQ ID NO: 73 - DNA, 10 bp, Artificial sequence
  Molecular barcode
tgccttccga

SEQ ID NO: 74 - DNA, 10 bp, Artificial sequence
  Molecular barcode
gcgacatcgg

SEQ ID NO: 75 - DNA, 10 bp, Artificial sequence
  Molecular barcode
cattctaagt

SEQ ID NO: 76 - DNA, 10 bp, Artificial sequence
  Molecular barcode
caggcttgga

SEQ ID NO: 77 - DNA, 10 bp, Artificial sequence
  Molecular barcode
atcatcgtct

SEQ ID NO: 78 - DNA, 10 bp, Artificial sequence
  Molecular barcode
gtcttgtgag

SEQ ID NO: 79 - DNA, 10 bp, Artificial sequence
  Molecular barcode
agtaggaacg

SEQ ID NO: 80 - DNA, 10 bp, Artificial sequence
  Molecular barcode
tcacaaccac

SEQ ID NO: 81 - DNA, 10 bp, Artificial sequence
  Molecular barcode
gcaggccttc

SEQ ID NO: 82 - DNA, 10 bp, Artificial sequence
  Molecular barcode
tggcaagcta

SEQ ID NO: 83 - DNA, 10 bp, Artificial sequence
  Molecular barcode
gagcattgtc

SEQ ID NO: 84 - DNA, 10 bp, Artificial sequence
  Molecular barcode
tgtgattagc

SEQ ID NO: 85 - DNA, 10 bp, Artificial sequence
  Molecular barcode
cctatggact

SEQ ID NO: 86 - DNA, 10 bp, Artificial sequence
  Molecular barcode
taggcgatag

SEQ ID NO: 87 - DNA, 10 bp, Artificial sequence
  Molecular barcode
agaccacgat

SEQ ID NO: 88 - DNA, 10 bp, Artificial sequence
  Molecular barcode
gtattagcca

SEQ ID NO: 89 - DNA, 10 bp, Artificial sequence
  Molecular barcode
ctctgcactg

SEQ ID NO: 90 - DNA, 10 bp, Artificial sequence
  Molecular barcode
accagcctga

SEQ ID NO: 91 - DNA, 10 bp, Artificial sequence
  Molecular barcode
gcgtgagtat

SEQ ID NO: 92 - DNA, 10 bp, Artificial sequence
  Molecular barcode
cgcggagcat

SEQ ID NO: 93 - DNA, 10 bp, Artificial sequence
  Molecular barcode
caagttcaca

SEQ ID NO: 94 - DNA, 10 bp, Artificial sequence
  Molecular barcode
agcacctctc

SEQ ID NO: 95 - DNA, 10 bp, Artificial sequence
  Molecular barcode
ttacagtgca

SEQ ID NO: 96 - DNA, 10 bp, Artificial sequence
  Molecular barcode
ttgcctaggc

SEQ ID NO: 97 - DNA, 10 bp, Artificial sequence
  Molecular barcode
gctatgatgg

SEQ ID NO: 98 - DNA, 10 bp, Artificial sequence
  Molecular barcode
aattaccatg

SEQ ID NO: 99 - DNA, 10 bp, Artificial sequence
  Molecular barcode
agacatggtg

SEQ ID NO: 100 - DNA, 10 bp, Artificial sequence
  Molecular barcode
ccagacatat

SEQ ID NO: 101 - DNA, 10 bp, Artificial sequence
  Molecular barcode
acgcttcctt

SEQ ID NO: 102 - DNA, 10 bp, Artificial sequence
  Molecular barcode
gacgtcttga

SEQ ID NO: 103 - DNA, 10 bp, Artificial sequence
  Molecular barcode
tactgagcgg

SEQ ID NO: 104 - DNA, 10 bp, Artificial sequence
  Molecular barcode
tgtacacacc

SEQ ID NO: 105 - DNA, 10 bp, Artificial sequence
  Molecular barcode
cttacgtgaa

SEQ ID NO: 106 - DNA, 10 bp, Artificial sequence
  Molecular barcode
gtgtggaacc

SEQ ID NO: 107 - DNA, 10 bp, Artificial sequence
  Molecular barcode
aagaatacct

SEQ ID NO: 108 - DNA, 10 bp, Artificial sequence
  Molecular barcode
gttgcattcg

SEQ ID NO: 109 - DNA, 10 bp, Artificial sequence
  Molecular barcode
cgccgttgaa

SEQ ID NO: 110 - DNA, 10 bp, Artificial sequence
  Molecular barcode
ttccgccgag

SEQ ID NO: 111 - DNA, 10 bp, Artificial sequence
  Molecular barcode
ccattaccgt

SEQ ID NO: 112 - DNA, 10 bp, Artificial sequence
  Molecular barcode
acgtcggatc

SEQ ID NO: 113 - DNA, 10 bp, Artificial sequence
  Molecular barcode
tgtatcgtga

SEQ ID NO: 114 - DNA, 10 bp, Artificial sequence
  Molecular barcode
gaagagaatc

SEQ ID NO: 115 - DNA, 10 bp, Artificial sequence
  Molecular barcode
cattaattct

SEQ ID NO: 116 - DNA, 10 bp, Artificial sequence
  Molecular barcode
tgacgctggt

SEQ ID NO: 117 - DNA, 10 bp, Artificial sequence
  Molecular barcode
gagcctgacg

SEQ ID NO: 118 - DNA, 10 bp, Artificial sequence
  Molecular barcode
ctagagcagg

SEQ ID NO: 119 - DNA, 10 bp, Artificial sequence
  Molecular barcode
agttgagtta

SEQ ID NO: 120 - DNA, 10 bp, Artificial sequence
  Molecular barcode
gcggtcacta

SEQ ID NO: 121 - DNA, 10 bp, Artificial sequence
  Molecular barcode
ttcactccac

SEQ ID NO: 122 - DNA, 10 bp, Artificial sequence
  Molecular barcode
accatgagac

SEQ ID NO: 123 - DNA, 10 bp, Artificial sequence
  Molecular barcode
taggttgttc

SEQ ID NO: 124 - DNA, 10 bp, Artificial sequence
  Molecular barcode
ctgactctgg

SEQ ID NO: 125 - DNA, 10 bp, Artificial sequence
  Molecular barcode
actgcctgtt

SEQ ID NO: 126 - DNA, 10 bp, Artificial sequence
  Molecular barcode
gtcatggagc

SEQ ID NO: 127 - DNA, 10 bp, Artificial sequence
  Molecular barcode
ggatagacat

SEQ ID NO: 128 - DNA, 10 bp, Artificial sequence
  Molecular barcode
cctcgacaag

SEQ ID NO: 129 - DNA, 10 bp, Artificial sequence
  Molecular barcode
taccgaagca

SEQ ID NO: 130 - DNA, 10 bp, Artificial sequence
  Molecular barcode
agatactcca

SEQ ID NO: 131 - DNA, 10 bp, Artificial sequence
  Molecular barcode
ttgatcaagg

SEQ ID NO: 132 - DNA, 10 bp, Artificial sequence
  Molecular barcode
tgccacttcc

SEQ ID NO: 133 - DNA, 10 bp, Artificial sequence
  Molecular barcode
gtagaatgtt

SEQ ID NO: 134 - DNA, 10 bp, Artificial sequence
  Molecular barcode
gactcgcgtc

SEQ ID NO: 135 - DNA, 10 bp, Artificial sequence
  Molecular barcode
agtgttatag

SEQ ID NO: 136 - DNA, 10 bp, Artificial sequence
  Molecular barcode
acacgagact

SEQ ID NO: 137 - DNA, 10 bp, Artificial sequence
  Molecular barcode
cataggccga

SEQ ID NO: 138 - DNA, 10 bp, Artificial sequence
  Molecular barcode
ccgtctgcaa

SEQ ID NO: 139 - DNA, 10 bp, Artificial sequence
  Molecular barcode
actcatacgc

(19) United States
(12) Patent Application Publication
(10) Pub. No.: US 2023/0174969 A1
(43) Pub. Date: Jun. 8, 2023

(54) BARCODED TRANSPOSASE COMPLEX AND APPLICATION THEREOF IN HIGH-THROUGHPUT SEQUENCING

(71) Applicant: MGI TECH CO., LTD., Guangdong (CN)

(72) Inventors: Xiaofang CHENG, Guangdong (CN); Yan ZOU, Guangdong (CN); Dan CHEN, Guangdong (CN); Zhou LONG, Guangdong (CN); Lei CAO, Guangdong (CN); Lin XIE, Guangdong (CN); Ping LIU, Guangdong (CN)

(21) Appl. No.: 17/925,157

(22) PCT Filed: May 18, 2020
(86) PCT No.: PCT/CN2020/090790

§ 371 Date: Nov. 14, 2022

(51) Int. Cl. C12N 15/10

(52) U.S. Cl. CPC C12N 15/1065

(57) ABSTRACT

A barcoded transposase complex and an application thereof in high-throughput sequencing. Provided is a transposase recognition element, having the following structure: X(m)Y(f)N(n), in which X(m) represents a transposase recognition region of a double-stranded nucleic acid structure, Y(f) represents a spacer region of a single-stranded DNA structure, and N(n) represents a sample barcode of a single-stranded DNA structure. The high-molecular-weight DNA is processed using the barcoded transposase complex, to obtain a lot of barcoded DNA fragments. The barcoded DNA fragments obtained from each high-molecular-weight DNA are mixed to obtain a mixing sample. A carrier having a molecular barcode is adopted to capture. An exonuclease is adopted for processing, and then transposase is released. StLFR technology is adopted to construct a DNA library. The barcoded transposase complex can be applied to hybrid sequencing of a high-throughput sequencing platform.

### TECHNICAL FIELD

The present disclosure relates to the field of biotechnology and, in particular, to a barcoded transposase complex and use thereof in high-throughput sequencing.

### BACKGROUND

With the continuous development of a new generation of sequencing technology, breakthroughs have been made in the field of genomics, bringing biological researches into an era of big data. With an increasingly wider application of biological data in public life, gene sequencing technology is widely used in the fields of birth defects, prevention and control of tumors and accurate diagnosis and treatment of contagious diseases and infectious diseases. The earliest first-generation sequencing played an important role in the Human Genome Project, but the expensive price was daunting. Powerful second-generation sequencing technologies such as a 454 sequencing system of Roche, a Solexa technology of Illumina, Inc., a Solid technology of ABI Companies, Inc. and a nanosphere sequencing technology of Beijing Genomics Institute (BGI) have reduced the cost of human genome sequencing by thousands of times. With the assistance of the second-generation sequencing technologies, a genome map is being drawn in full swing, and at the same time, a third-generation sequencing technology, a strong opponent of the second-generation sequencing technology, has temporarily emerged. However, at present, the third-generation sequencing technology still has the problems such as a high requirement for sample and a high sequencing cost, and it is difficult to occupy even half of the market. Due to a low cost and high throughput, the second-generation sequencing technology is most widely applied. However, since the traditional second-generation sequencing library is limited by factors such as short read lengths and small span and a piece of very important information (haplotype information) is ignored so that more accurate genomic information cannot be obtained.

In this context, MGI has independently developed a new generation of long-fragment DNA library construction technology, single-tube long fragment read (stLFR). This technology is based on a DNA molecule partition-less co-barcoding technology of a patent developed by MGI. Long-length information is obtained by using high-precision short reads, integrating advantages of the second-generation sequencing and the third-generation sequencing. The partition-less co-barcoding technology of the stLFR is as follows. Tens of millions of virtual compartments are formed on a surface of a magnetic bead, where different virtual compartments carry different molecular barcodes, a limited amount of high-molecular-weight DNA is separately placed in the same reaction tube and reacted with an enzyme to fragment the high-molecular-weight DNA, and DNA in the same compartment is labeled with the same molecular barcode through the virtual compartments. Through molecular barcode information, long-length information is generated using the short read sequences obtained through sequencing, so as to obtain phasing information of a heterozygous site on a diploid genome, with a phased region N50 value reaching more than or equal to 10 Mb, achieving long-fragment information applications such as a high-quality variation detection and a structural variation analysis. At present, this technology is a simplest method for sequencing a haploid genome with a very low requirement for sample, a starting amount of only 1.5 ng and no pre-amplification required. Moreover, no complex pipetting device or microfluidic device is required to perform physical separation, and all reactions are performed in a single reaction tube and completed on magnetic beads, which is easy to achieve high-throughput automation and significantly reduces the complexity and cost of constructing a long-fragment library. This technology is widely applied in the fields of individual genomes, researches on complex diseases, researches on tumor genomes, assembly and resequencing of animal and plant genomes and assembly and resequencing of microbial genomes.

In the stLFR technology provided by MGI, tens of millions of virtual compartments are formed on the surface of the magnetic bead, and a transposase is used for fragmenting the high-molecular-weight DNA. The virtual compartments make the short fragments in the same compartment carry the same molecular barcode, and the phased region of the diploid is as long as 10 Mb. At present, the stLFR technology performs library construction and sequencing on a single sample and cannot achieve mixed library construction and sequencing for large samples, resulting in a waste of sequencing resources and costs for stLFR library construction and sequencing of some small genomic samples or samples that do not require too much amount of data. With the continuous improvement of sequencing throughput of a sequencing instrument, a higher requirement is also imposed on throughput of the library construction, and a large-sample mixed library construction technology is a general trend.

### SUMMARY

The present disclosure provides a barcoded transposase complex and use thereof in high-throughput sequencing.

The present disclosure provides a transposase recognition element, which is characterized by the following (a) and/or (b):

(a) a transferred strand contains a fixed sequence;

(b) a non-transferred strand contains a U base.

The present disclosure provides a transposase recognition element, which is characterized in that:

the transposase recognition element has a structure of X(m)Y(f)N(n); where

X(m) denotes a transposase recognition region and has a double-stranded nucleic acid structure;

Y(f) denotes a spacer region and has a single-stranded DNA structure; and

N(n) denotes a sample barcode and has a single-stranded DNA structure.

In the transposase recognition region, a portion of T in one strand is replaced with U.

One strand of X(m) consists of A, T, C and G, and the other strand of X(m) consists of A, T, C, G and U.

X(m) has a size of 19 bp.

Y(f) has a size of 15-30 nt (may specifically be 20 nt).

N(n) has a size of 8-12 nt (may specifically be 10 nt).

Each nucleotide in N(n) is any one of A, T, C and G.

A transposase recognition element is specifically formed of a single-stranded nucleic acid molecule A1 and a single-stranded nucleic acid molecule A2.

A transposase recognition element is specifically formed of a single-stranded nucleic acid molecule A1 and a single-stranded nucleic acid molecule C.

The single-stranded nucleic acid molecule A1 is shown in Sequence 1 in the sequence list.

The single-stranded nucleic acid molecule A2 is shown in Sequence 2 in the sequence list.

The single-stranded nucleic acid molecule C is shown in Sequence 9 in the sequence list.

Specifically, each sample barcode listed in Table 1 may be used.

The present disclosure provides a barcoded transposase complex. The barcoded transposase complex is formed of a transposase and any one of the above transposase recognition elements. The barcoded transposase complex is formed through co-incubation and self-assembly of a transposase and any one of the above transposase recognition elements.

The present disclosure provides a method (a method I) for preparing a barcoded DNA fragment. The method includes the following steps: providing high-molecular-weight DNA and treating with the barcoded transposase complex.

The present disclosure provides a method (a method II) for constructing a DNA library. The method includes the following steps in sequence:

(1) providing high-molecular-weight DNA and preparing a barcoded DNA fragment using the method I; and

(2) treating with an exonuclease and releasing the transposase.

The present disclosure provides a method (a method III) for constructing a DNA library.

The method includes the following steps in sequence:

(1) providing high-molecular-weight DNA and preparing a barcoded DNA fragment using the method I;

(2) capturing with a carrier containing a molecular barcode; and

(3) treating with an exonuclease and releasing the transposase.

The present disclosure provides a method (a method IV) for constructing a DNA library (a multi-sample mixed sequencing library). The method includes the following steps in sequence:

(1) providing n pieces of high-molecular-weight DNA and preparing barcoded DNA fragments using the method I, respectively, where n is a natural number greater than or equal to 2;

(2) mixing the barcoded DNA fragments obtained after each high-molecular-weight DNA is subjected to step (1), to obtain a mixed sample; and

(3) treating with an exonuclease and releasing the transposase.

The method IV further includes the following step:

(4) performing library construction using an stLFR technology to obtain the DNA library.

The present disclosure provides a method (a method V) for constructing a DNA library (a multi-sample mixed sequencing library). The method includes the following steps in sequence:

(1) providing n pieces of high-molecular-weight DNA and preparing barcoded DNA fragments using the method I, respectively, where n is a natural number greater than or equal to 2;

(2) mixing the barcoded DNA fragments obtained after each high-molecular-weight DNA is subjected to step (1), to obtain a mixed sample;

(3) capturing the mixed sample obtained in step (2) with a carrier containing a molecular barcode; and

(4) treating with an exonuclease and releasing the transposase.

The method V further includes the following step:

(5) performing library construction using an stLFR technology to obtain the DNA library.

The present disclosure provides a kit for preparing a barcoded DNA fragment. The kit includes a transposase and any one of the above transposase recognition elements.

The present disclosure provides a kit for preparing a barcoded DNA fragment. The kit includes the barcoded transposase complex.

The present disclosure provides a kit for constructing a DNA library. The kit includes a transposase and any one of the above transposase recognition elements. The kit further includes an exonuclease. The kit further includes a carrier containing a molecular barcode.

The present disclosure provides a kit for constructing a DNA library. The kit includes the barcoded transposase complex. The kit further includes an exonuclease. The kit further includes a carrier containing a molecular barcode.

Any one of the above transposases may specifically be a Tn5 transposase.

Any one of the above exonucleases may specifically be an exonuclease I and an exonuclease III.

Any one of the above release of the transposases is achieved through the addition of a denaturing agent. The denaturing agent may specifically be sodium dodecyl sulfate (SDS).

Performing the library construction using the stLFR technology includes the steps of adding an adapter, polymerase chain reaction (PCR) amplification and PCR purification.

The adapter consists of a single-stranded DNA molecule adapter-1A and a single-stranded DNA molecule adapter-2A. The single-stranded DNA molecule adapter-1A is shown in Sequence 5 in the sequence list. The single-stranded DNA molecule adapter-2A is shown in Sequence 6 in the sequence list.

A pair of primers consisting of primer-F and primer-R is used for the PCR amplification.

Primer-F is shown in Sequence 7 in the sequence list. Primer-R is shown in Sequence 8 in the sequence list.

The high-molecular-weight DNA, also known as long-fragment DNA, is more than or equal to 40 Kb.

For example, the high-molecular-weight DNA may be genomic DNA obtained through DNA extraction of a biological sample.

The high-molecular-weight DNA is treated using the barcoded transposase complex to obtain a large number of barcoded DNA fragments, where each of the fragments has a size of 200-2000 bp. For each high-molecular-weight DNA, the used barcoded transposase complex contains a unique sample barcode so that the barcoded DNA fragments derived from each high-molecular-weight DNA contain the unique sample barcode and all the barcoded DNA fragments derived from each high-molecular-weight DNA contain the same sample barcode.

The carrier containing a molecular barcode is high-throughput hybridization capture sequence-contained magnetic bead carriers (the high-throughput magnetic bead carriers include a very large number types of hybridization capture sequence-contained magnetic bead carriers).

The hybridization capture sequence-contained magnetic bead carrier is a magnetic bead to which a specific nucleic acid molecule has been attached. The specific nucleic acid molecule has a partially double-stranded structure. A segment at one end of a first strand is reverse complementary to a segment at one end of a second strand to form the partially double-stranded structure. The first strand is attached to the magnetic bead at its free end, and contains a molecular barcode (located in a non-double-stranded structure of the specific nucleic acid molecule) in the strand. The second strand contains a transposon capture region (located in the non-double-stranded structure of the specific nucleic acid molecule, where the transposon capture region is reverse complementary to a capture recognition region) at its free end.

Each magnetic bead contains multiple specific nucleic acid molecules that are the same (that is, all the specific nucleic acid molecules on each magnetic bead contain the same molecular barcode). For all hybridization capture sequence-contained magnetic bead carriers, other moieties of the specific nucleic acid molecules are the same except for the sequence of the molecular barcode. Hybridization capture sequence-contained magnetic bead carriers that contain the same specific nucleic acid molecule (that is, contain the same molecular barcode) are considered as one type of hybridization capture sequence-contained magnetic bead carrier.

The hybridization capture sequence-contained magnetic bead carrier is a magnetic bead to which a specific nucleic acid molecule has been attached. The specific nucleic acid molecule consists of a single-stranded nucleic acid molecule B1 and a single-stranded nucleic acid molecule B2 and has a partially double-stranded structure. The 5′-end of the single-stranded nucleic acid molecule B1 is attached to the magnetic bead. A 3′-end segment of the single-stranded nucleic acid molecule B1 is reverse complementary to a 3′-end segment of the single-stranded nucleic acid molecule B2 to form the partially double-stranded structure. The single-stranded nucleic acid molecule B1 contains molecular barcode 1, molecular barcode 2 and molecular barcode 3 (located in a non-double-stranded structure of the specific nucleic acid molecule). In the single-stranded nucleic acid molecule B1, the 5′-end sequence is shown in Sequence 3 in the sequence list (located upstream of the three molecular barcodes). In the single-stranded nucleic acid molecule B2, the 5′-end contains a transposon capture region (located in a non-double-stranded structure of the specific nucleic acid molecule, where the transposon capture region is reverse complementary to the capture recognition region). The single-stranded nucleic acid molecule B2 is shown in Sequence 4 in the sequence list. Each of the molecular barcode 1, the molecular barcode 2 and the molecular barcode 3 consists of ten nucleotides, where each nucleotide is any one of A, T, C and G.

Since the transposase is not subjected to denaturation treatment, the transposase still retains the integrity of the DNA while occupying and protecting an enzyme digestion recognition site of the DNA. Moreover, only 1% of oligonucleotides on a magnetic bead modified with a large number of oligonucleotides with the same sequence can be used for binding to the DNA, and remaining 99% exposed oligonucleotides will participate in subsequent adapter ligation and PCR to compete with a real product. Therefore, the excess oligonucleotides on surface of the magnetic bead should be cleaved using exonuclease, while protecting the inserted DNA fragment from enzyme digestion of the exonuclease.

After the enzyme digestion, a denaturing agent for transposase is added to terminate the action of the exonuclease while denaturing the transposase so that the transposase is completely released from the DNA.

The DNA library is taken and subjected to high-throughput sequencing. Then, sequencing results are attributed to each sample through the sample barcode, and short read length sequences generated through sequencing are spliced into original long-fragment DNA information through molecular barcode information carried on the stLFR magnetic bead, achieving haplotype sequencing.

The present disclosure also protects use of any one of the above transposase recognition elements in DNA sequencing.

The present disclosure also protects use of the above barcoded transposase complex in DNA sequencing.

The present disclosure also protects use of any one of the above methods in DNA sequencing.

The present disclosure also protects use of any one of the above kits in DNA sequencing.

Any of the above sequencing is haploid sequencing.

In view of a deficiency that the stLFR can only perform library construction and sequencing on a single sample at present, based on the stLFR technology, the present disclosure provides a solution suitable for mixed library construction of a large number of samples.

A structure diagram of a barcoded transposase-loading element is shown in FIG. 1. A structure diagram of a barcoded transposase complex is shown in FIG. 2.

Main inventive points of the method of the present disclosure are described below.

(1) A barcoded transposase-loading element and a barcoded transposase complex are designed. In the barcoded transposase-loading element, a spacer region is disposed between a transposase recognition region and a sample barcode. A sequence pool of the sample barcodes is designed (Table 1).

(2) After the high-molecular-weight DNA (greater than 40 Kb) is fragmented and barcoded using the barcoded transposase, the barcoded DNA fragments are subjected to sample mixing without releasing the transposase (not subjected to denaturation treatment) before hybridization capture. For the subsequent step of enzyme digestion, the transposase provides space-occupying protection for the inserted DNA fragment, that is, protects the inserted DNA fragment from being recognized and cleaved by the exonuclease, and only the oligonucleotides exposed on the surface of the magnetic bead is cleaved by the exonuclease. On the one hand, the loss of effective data and diversity caused by the loss of samples during library construction is reduced, which is conducive to improving the uniformity of coverage. On the other hand, the complexity of the operation of library construction is reduced, and throughput of the library construction is improved, which is conductive to maximizing the utilization of throughput of a sequencing instrument and saving the time and costs of the library construction and sequencing for a single sample.

Compared with the existing art, the present disclosure has the following advantages: (1) mixed library construction may be performed on a large number of samples, reducing the complexity and cost of stLFR library construction for a single sample; (2) the multiple samples are mixed before magnetic bead hybridization capture, further improving the throughput of the library construction; (3) the utilization rate of stFLR capture beads in the step of hybridization capture is improved so that multiple samples are captured on one magnetic bead and the multiple samples do not interfere with each other; (4) the utilization rate of sequencing throughput is improved, and the sequencing cost is reduced; (5) high-throughput automated library construction is convenient to be achieved; (6) the present disclosure is applicable to small genomic samples and samples with a requirement for a specific amount of data, and resequencing and de novo assembly of long-fragment information are obtained based on a short sequencing read length; and (7) based on that stLFR only requires 1.5 ng to start, the initial input of a single sample may be further reduced, which is applicable to sequencing researches on rare and very low biomass samples.

The present disclosure has the following beneficial effects: (1) the present disclosure provides a stLFR-based multi-sample mixed library construction technology, which successfully solves the problems of mixed library construction and sequencing of large samples; (2) the present disclosure may significantly reduce the complexity of library construction, improve throughput of the library construction, improve a utilization rate of a sequencing instrument and reduce costs of library construction and sequencing for a single sample; (3) the present disclosure is applicable to resequencing and de novo assembly of samples with a small genome and samples with a requirement for a specific amount of data; (4) the present disclosure may further reduce an initial starting amount of a single sample to less than 1.5 ng, which is applicable to resequencing and de novo assembly of rare samples and samples in very low biomass; and (5) high-throughput automated library construction is convenient to be achieved.

### BRIEF DESCRIPTION OF DRAWINGS

FIG. 1 is a structure diagram of elements of a barcoded transposase-loading fragment.

FIG. 2 is a structure diagram of a barcoded transposase complex.

FIG. 3 is a structure diagram of elements of a hybridization capture sequence-contained magnetic bead carrier.

FIG. 4 is a flowchart of library construction.

FIG. 5 is an electrophoresis diagram of step 11 according to Example 2.

FIG. 6 illustrates results of quantification using a Qubit™ double-stranded DNA high-sensitivity fluorescence quantification kit and calculation of a polymerase chain reaction (PCR) yield in Example 3.

FIG. 7 is a diagram illustrating results of electrophoresis detection in Example 3.

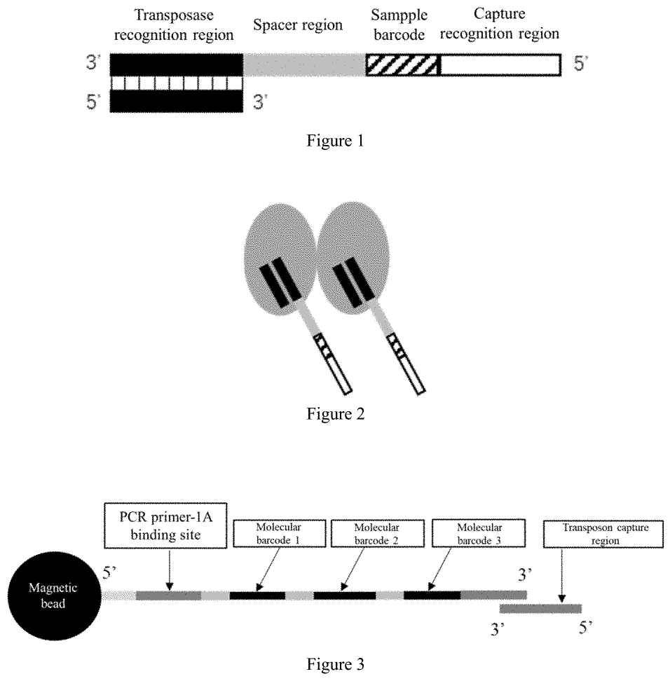

### DETAILED DESCRIPTION

The following examples facilitate a better understanding of the present disclosure and do not limit the present disclosure. The experimental methods in the following examples are conventional methods unless otherwise specified. The experimental materials used in the following examples are purchased from conventional biochemical reagent stores unless otherwise specified. The quantitative experiments in the following examples are all provided with three repeated experiments, and the results are averaged. Unless otherwise specified, among the nucleic acid molecules in the examples, A refers to an adenine deoxyribonucleotide, C refers to a cytosine deoxyribonucleotide, G refers to a guanine deoxyribonucleotide, T refers to a thymine deoxyribonucleotide, and U refers to a uracil ribonucleotide.

### Example 1. Establishment of Method

Transposase, a commonly used tool enzyme for next-generation library construction, can achieve rapid fragmentation of DNA. In the present disclosure, a barcoded transposase-loading fragment is designed and prepared. The barcoded transposase-loading fragment is self-assembled with a transposase to form a barcoded transposase complex, and when the barcoded transposase complex is subjected to a transposition reaction, high-molecular-weight DNA is fragmented and barcoded. Further, after the transposition reaction is performed, the transposase is not subjected to denaturation treatment and retains the integrity of the nucleic acid molecule fragments while occupying and protecting enzyme digestion recognition sites of the nucleic acid molecule fragments, protecting the nucleic acid molecule fragments from an action of an exonuclease.

1. Preparation of barcoded DNA fragments

(1) Preparation of high-molecular-weight DNA

The high-molecular-weight DNA, also known as long-fragment DNA, is commonly greater than 40 Kb.

For example, the high-molecular-weight DNA may be genomic DNA obtained through DNA extraction of a biological sample.

(2) Preparation of a barcoded transposase-loading fragment

The barcoded transposase-loading fragment has a structure of X(m)Y(f)N(n).

X(m) denotes a transposase recognition region, which has a double-stranded nucleic acid structure (one strand consists of A, T, C and G, and the other strand consists of A, T, C, G and U) and a size of 19 bp.

Y(f) denotes a spacer region, which has a single-stranded DNA structure and a size of 15-30 nt (may specifically be 20 nt). The spacer region is used for separating the transposase recognition region and a sample barcode (reducing a direct effect of the sample barcode on the transposase) and may also be used for designing sequencing primers in a subsequent process.

N(n) denotes the sample barcode, which has a single-stranded DNA structure and a size of 8-12 nt (may specifically be 10 nt), where each nucleotide is any one of A, T, C and G. Each sample corresponds to a unique sample barcode for distinguishing a source of the sample.

Specifically, each sample barcode listed in Table 1 (in Table 1, the sequences are all in a 5′ →3′ direction) may be used.

TABLE 1

| Name | Sequence |
|---|---|
| Molecular | ATCGGACCTA |
| barcode 01 | |
| Molecular | GATTCCGTCC |
| barcode 02 | |
| Molecular | CGGCAGTAAG |
| barcode 03 | |
| Molecular | TCAATTAGGT |
| barcode 04 | |
| Molecular | CGGATACGAA |
| barcode 05 | |
| Molecular | GCTCGTTACC |
| barcode 06 | |
| Molecular | TTATACGTTG |
| barcode 07 | |
| Molecular | AACGCGACGT |
| barcode 08 | |
| Molecular | GCTAGCAGAA |
| barcode 09 | |
| Molecular | CTATCTTCCT |
| barcode 10 | |
| Molecular | AAGCAAGAGC |
| barcode 11 | |
| Molecular | TGCGTGCTTG |
| barcode 12 | |
| Molecular | CGGATTGCCG |
| barcode 13 | |
| Molecular | GAATCCTGAT |
| barcode 14 | |
| Molecular | TCTGGAATGA |
| barcode 15 | |
| Molecular | ATCCAGCATC |
| barcode 16 | |
| Molecular | CATCACTCAC |
| barcode 17 | |
| Molecular | CAGCTGACTC |
| barcode 18 | |
| Molecular | TTCGCAGACA |
| barcode 19 | |
| Molecular | TTGTACCAAT |
| barcode 20 | |
| Molecular | ACCACAATCG |
| barcode 21 | |
| Molecular | GGAAGTCTGT |
| barcode 22 | |
| Molecular | AGAGTGTGGA |
| barcode 23 | |
| Molecular | GCTTGTGGTG |
| barcode 24 | |
| Molecular | TTGTCCTCTA |
| barcode 25 | |
| Molecular | ATTCGCTAGG |
| barcode 26 | |
| Molecular | CGATGACTAC |
| barcode 27 | |
| Molecular | ACAGCTCAGC |
| barcode 28 | |
| Molecular | TATCTAGGTT |
| barcode 29 | |
| Molecular | GAGATGGCAA |
| barcode 30 | |
| Molecular | CGCAAGATCT |
| barcode 31 | |
| Molecular | GCCGATAGCG |
| barcode 32 | |
| Molecular | CCATCGTTGC |
| barcode 33 | |
| Molecular | TGAACGATTA |
| barcode 34 | |
| Molecular | TAGAGCGAAC |
| barcode 35 | |
| Molecular | ATGTGTGAGA |
| barcode 36 | |
| Molecular | ATCCTAACAG |
| barcode 37 | |
| Molecular | CGCGTCTGCG |
| barcode 38 | |
| Molecular | GATGATCCTT |
| barcode 39 | |
| Molecular | GCTCAACGCT |
| barcode 40 | |
| Molecular | ATGCATCTAA |
| barcode 41 | |
| Molecular | AGCTCTGGAC |
| barcode 42 | |
| Molecular | CTATCACGTG |
| barcode 43 | |
| Molecular | GGACTAGTGG |
| barcode 44 | |
| Molecular | GCCAAGTCCA |
| barcode 45 | |
| Molecular | CCTGTCAAGC |
| barcode 46 | |
| Molecular | TAGAGGTCTT |
| barcode 47 | |
| Molecular | TATGGCAACT |
| barcode 48 | |
| Molecular | CTGCGTACAT |
| barcode 49 | |
| Molecular | ATCTCATTAA |
| barcode 50 | |
| Molecular | AAGTGGCGCA |
| barcode 51 | |
| Molecular | GGCCTTAATG |
| barcode 52 | |
| Molecular | TCTGAGGCGG |
| barcode 53 | |
| Molecular | CGAGCCGATT |
| barcode 54 | |
| Molecular | GATAACCGGC |
| barcode 55 | |
| Molecular | TCAATATTCC |
| barcode 56 | |
| Molecular | TCCGTTGAAT |
| barcode 57 | |
| Molecular | CAGTACAGTT |
| barcode 58 | |
| Molecular | ATTGAGGTAC |
| barcode 59 | |
| Molecular | ATTAGAAGTC |
| barcode 60 | |
| Molecular | CAACGCTTCA |
| barcode 61 | |
| Molecular | GGATCGCACG |
| barcode 62 | |
| Molecular | TGCCTTCCGA |
| barcode 63 | |
| Molecular | GCGACATCGG |
| barcode 64 | |
| Molecular | CATTCTAAGT |
| barcode 65 | |
| Molecular | CAGGCTTGGA |
| barcode 66 | |
| Molecular | ATCATCGTCT |
| barcode 67 | |
| Molecular | GTCTTGTGAG |
| barcode 68 | |
| Molecular | AGTAGGAACG |
| barcode 69 | |
| Molecular | TCACAACCAC |
| barcode 70 | |
| Molecular | GCAGGCCTTC |
| barcode 71 | |
| Molecular | TGGCAAGCTA |
| barcode 72 | |
| Molecular | GAGCATTGTC |
| barcode 73 | |
| Molecular | TGTGATTAGC |
| barcode 74 | |
| Molecular | CCTATGGACT |
| barcode 75 | |
| Molecular | TAGGCGATAG |
| barcode 76 | |
| Molecular | AGACCACGAT |
| barcode 77 | |
| Molecular | GTATTAGCCA |
| barcode 78 | |
| Molecular | CTCTGCACTG |
| barcode 79 | |
| Molecular | ACCAGCCTGA |
| barcode 80 | |
| Molecular | GCGTGAGTAT |
| barcode 81 | |
| Molecular | CGCGGAGCAT |
| barcode 82 | |
| Molecular | CAAGTTCACA |
| barcode 83 | |
| Molecular | AGCACCTCTC |
| barcode 84 | |
| Molecular | TTACAGTGCA |
| barcode 85 | |
| Molecular | TTGCCTAGGC |
| barcode 86 | |
| Molecular | GCTATGATGG |
| barcode 87 | |
| Molecular | AATTACCATG |
| barcode 88 | |
| Molecular | AGACATGGTG |
| barcode 89 | |
| Molecular | CCAGACATAT |
| barcode 90 | |
| Molecular | ACGCTTCCTT |
| barcode 91 | |
| Molecular | GACGTCTTGA |
| barcode 92 | |
| Molecular | TACTGAGCGG |
| barcode 93 | |
| Molecular | TGTACACACC |
| barcode 94 | |
| Molecular | CTTACGTGAA |
| barcode 95 | |
| Molecular | GTGTGGAACC |
| barcode 96 | |
| Molecular | AAGAATACCT |
| barcode 97 | |
| Molecular | GTTGCATTCG |
| barcode 98 | |
| Molecular | CGCCGTTGAA |
| barcode 99 | |
| Molecular | TTCCGCCGAG |
| barcode 100 | |
| Molecular | CCATTACCGT |
| barcode 101 | |
| Molecular | ACGTCGGATC |
| barcode 102 | |
| Molecular | TGTATCGTGA |
| barcode 103 | |
| Molecular | GAAGAGAATC |
| barcode 104 | |
| Molecular | CATTAATTCT |
| barcode 105 | |
| Molecular | TGACGCTGGT |
| barcode 106 | |
| Molecular | GAGCCTGACG |
| barcode 107 | |
| Molecular | CTAGAGCAGG |
| barcode 108 | |
| Molecular | AGTTGAGTTA |
| barcode 109 | |
| Molecular | GCGGTCACTA |
| barcode 110 | |
| Molecular | TTCACTCCAC |
| barcode 111 | |
| Molecular | ACCATGAGAC |
| barcode 112 | |
| Molecular | TAGGTTGTTC |
| barcode 113 | |
| Molecular | CTGACTCTGG |
| barcode 114 | |
| Molecular | ACTGCCTGTT |
| barcode 115 | |
| Molecular | GTCATGGAGC |
| barcode 116 | |
| Molecular | GGATAGACAT |
| barcode 117 | |
| Molecular | CCTCGACAAG |
| barcode 118 | |
| Molecular | TACCGAAGCA |
| barcode 119 | |
| Molecular | AGATACTCCA |
| barcode 120 | |
| Molecular | TTGATCAAGG |
| barcode 121 | |
| Molecular | TGCCACTTCC |
| barcode 122 | |
| Molecular | GTAGAATGTT |
| barcode 123 | |
| Molecular | GACTCGCGTC |
| barcode 124 | |
| Molecular | AGTGTTATAG |
| barcode 125 | |
| Molecular | ACACGAGACT |
| barcode 126 | |
| Molecular | CATAGGCCGA |
| barcode 127 | |
| Molecular | CCGTCTGCAA |
| barcode 128 | |
| Molecular | ACTCATACGC |
| barcode 129 | |

(3) The barcoded transposase-loading fragment is co-incubated with a transposase to obtain a barcoded transposase complex.

(4) The high-molecular-weight DNA obtained in step (1) is fragmented and barcoded using the barcoded transposase complex obtained in step (3) to obtain a large number of barcoded DNA fragments, where each of the fragments has a size of 200-2000 bp. For each high-molecular-weight DNA, the used barcoded transposase complex contains a unique sample barcode so that the barcoded DNA fragments derived from each high-molecular-weight DNA contain the unique sample barcode and all the barcoded DNA fragments derived from each high-molecular-weight DNA contain the same sample barcode.

Note: after step (4) is completed, the transposase is not released.

2. Sample mixing before hybridization capture

The products obtained after each high-molecular-weight DNA is subjected to step 1 are mixed to obtain a mixed sample.

3. Hybridization capture of the barcoded DNA fragments

The mixed sample obtained in step 2 is taken and mixed with a high-throughput hybridization capture sequence-contained magnetic bead carrier (the high-throughput magnetic bead carrier includes a very large number types of hybridization capture sequence-contained magnetic bead carriers), and the hybridization capture sequence-contained magnetic bead carrier captured the barcoded DNA fragments through hybridization of DNA sequences.

The hybridization capture sequence-contained magnetic bead carrier is a magnetic bead to which a specific nucleic acid molecule has been attached. The specific nucleic acid molecule has a partially double-stranded structure. A segment at one end of a first strand is reverse complementary to a segment at one end of a second strand to form the partially double-stranded structure. The first strand is attached to the magnetic bead at its free end, and contains a molecular barcode (located in a non-double-stranded structure of the specific nucleic acid molecule) in the strand. The second strand contains a transposon capture region (located in the non-double-stranded structure of the specific nucleic acid molecule, where the transposon capture region is reverse complementary to a capture recognition region) at its free end.

Each magnetic bead contains multiple specific nucleic acid molecules that are the same (that is, all the specific nucleic acid molecules on each magnetic bead contain the same molecular barcode). For all hybridization capture sequence-contained magnetic bead carriers, other moieties of the specific nucleic acid molecules are the same except for the sequence of the molecular barcode. Hybridization capture sequence-contained magnetic bead carriers that contain the same specific nucleic acid molecule (that is, contain the same molecular barcode) are considered as one type of hybridization capture sequence-contained magnetic bead carrier.

4. Removing excess oligonucleotides on the magnetic bead through enzyme digestion

Since the transposase in step 3 is not subjected to denaturation treatment, the transposase still retains the integrity of the DNA while occupying and protecting an enzyme digestion recognition site of the DNA. Moreover, only 1% of oligonucleotides on a magnetic bead modified with a large number of oligonucleotides with the same sequence can be used for binding to the DNA, and remaining 99% exposed oligonucleotides will participate in subsequent adapter ligation and PCR to compete with a real product. Therefore, the excess oligonucleotides on surface of the magnetic bead should be cleaved using exonuclease, while protecting the inserted DNA fragment from enzyme digestion of the exonuclease.

After the enzyme digestion, a denaturing agent for transposase is added to terminate the action of the exonuclease while denaturing the transposase so that the transposase is completely released from the DNA.

5. The product in step 4 is taken, and library construction is performed using an stLFR technology to obtain a DNA library.

6. The DNA library obtained in step 5 is taken and subjected to high-throughput sequencing. Then, sequencing results are attributed to each sample through the sample barcode, and short read length sequences generated through sequencing are spliced into original long-fragment DNA information through molecular barcode information carried on the stLFR magnetic bead, achieving haplotype sequencing.

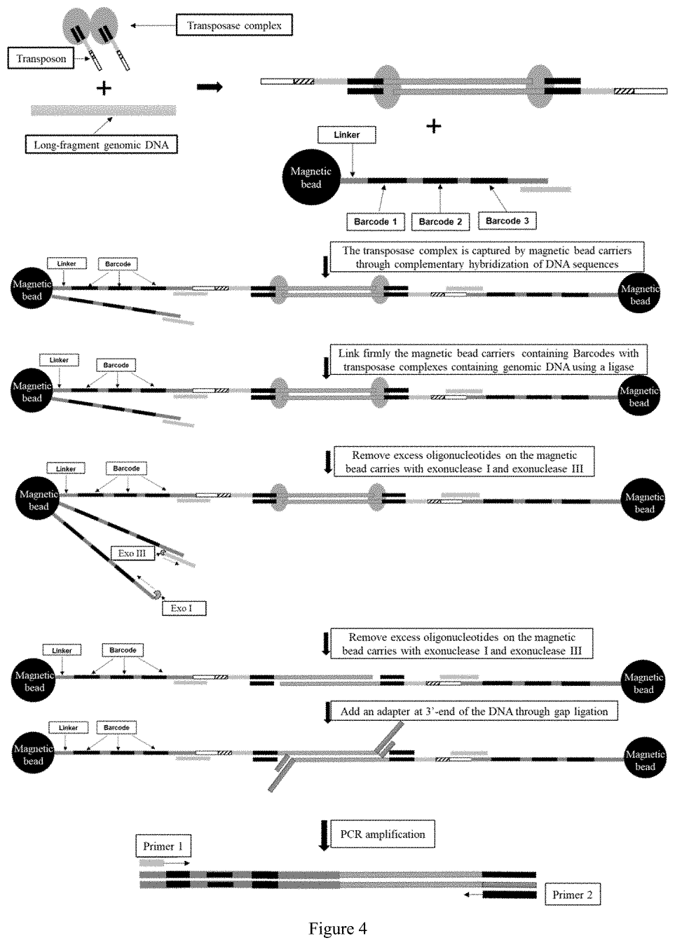

A flowchart of the library construction is shown in FIG. 4.

### Example 2. Specific Application of the Method

1. Preparation of a barcoded transposase complex

(1) Preparation of a barcoded transposase-loading fragment

The barcoded transposase-loading fragment was formed of a single-stranded nucleic acid molecule A1 and a single-stranded nucleic acid molecule A2.

The barcoded transposase-loading fragment was prepared by a specific method as follows: the single-stranded nucleic acid molecule A1 and the single-stranded nucleic acid molecule A2 (both at a concentration of 100 μM) are mixed in equal volumes and subjected to annealing to obtain a product solution. Annealing parameters: at 70° C. for 3 min; cooled to 20° C. (at a cooling rate of 0.1° C./s), at 20° C. for 30 min, and held at 4° C. The product solution contained the barcoded transposase-loading fragment at a concentration of 50 μM.

Single-strandednucleic acid molecule
A1 (Sequence 1):
5′Phos-CGATCCTTGGTGATCNNNNNNNNNN [symbol] [symbol]
AGATGTGTATAAGAGACAG-3′.
Single-strandednucleicacid molecule
A2 (Sequence 2):
5′PhOS-CTGUCTCUTATACACAUCT-3′.

In the single-stranded nucleic acid molecule A1, 10 N underlined by the straight line constituted a sample barcode, where N represented any one of A, T, C and G. Each sample corresponded to a unique sample barcode for distinguishing a source of the sample.

The bold moiety of the single-stranded nucleic acid molecule A1 and the single-stranded nucleic acid molecule A2 formed a double-stranded structure (the double-stranded structure was a transposase recognition region), and the remaining moiety was a single-stranded structure. The moiety underlined by the squiggle of the single-stranded nucleic acid molecule A1 was a spacer region, and the italic moiety of the single-stranded nucleic acid molecule A1 was a capture recognition region.

(2) Preparation of the barcoded transposase complex

16.52 μl of Tn5 transposase (purchased from BGI, Cat. No. BGE005, with a concentration of 1 U/μl), 17.08 μl of coupling buffer (6.3±0.1 g glycerol dissolved in 5 ml TE buffer), 17.92 μl of TE buffer and 4.48 μl of the product solution obtained in step (1) were uniformly mixed on ice and incubated at 30° C. for 1 h to obtain a product solution. The product solution was stored at −20° C. until use. The product solution contained the barcoded transposase complex.

2. Fragmentation and barcoding of high-molecular-weight DNA

The high-molecular-weight DNA was: NA12878 (CORIELL, Cat. No. NA12878), genomic DNA of Escherichia coli DH5α, genomic DNA of Arabidopsis lyrata, and Lambda DNA (ThermoFisher, Cat. No. SD0011), respectively.

10 ng of high-molecular-weight DNA was taken and added to a 0.2 ml centrifuge tube, and nuclease-free water was added to 36.8 μl. Then, 10 μl of 5×tagmentation buffer (purchased from BGI, Cat. No. BGE005B01) and 3.2 μl of 16-fold diluent (prepared by diluting the product solution obtained in (2) of step 1 to 16-fold volume with TE buffer, which was performed on ice) were added, uniformly mixed and incubated at 55° C. for 10 min to obtain a product solution. The 0.2 ml centrifuge tube containing the product solution was transferred to ice. The product solution contained barcoded DNA fragments.

For each type of high-molecular-weight DNA, the barcoded transposase complex used in the above steps contained a unique sample barcode so that the obtained barcoded DNA fragments contained the unique sample barcode.

3. Preparation of hybridization capture sequence-contained magnetic bead carrier

The hybridization capture sequence-contained magnetic bead carrier was a magnetic bead to which a specific nucleic acid molecule had been attached. The specific nucleic acid molecule consisted of a single-stranded nucleic acid molecule B1 and a single-stranded nucleic acid molecule B2 and had a partially double-stranded structure. The 5′-end of the single-stranded nucleic acid molecule B1 was attached to the magnetic bead. A 3′-end segment of the single-stranded nucleic acid molecule B1 was reverse complementary to a 3′-end segment of the single-stranded nucleic acid molecule B2 to form the partially double-stranded structure. The single-stranded nucleic acid molecule B1 contained molecular barcode 1, molecular barcode 2 and molecular barcode 3 (located in a non-double-stranded structure of the specific nucleic acid molecule). In the single-stranded nucleic acid molecule B1, the 5′-end sequence (Sequence 3) was AAAAAAAAAATGTGAGCCAAGGAGTTG (located upstream of the three molecular barcodes). In the single-stranded nucleic acid molecule B2, the 5′-end contained a transposon capture region (located in the non-double-stranded structure of the specific nucleic acid molecule, where the transposon capture region was reverse complementary to the capture recognition region).

Single-stranded nucleic acid molecule
B2 (Sequence 4):
5′- [symbol] CCATAGTCCATGCTA-3′.

The region underlined by the straight line of the single-stranded nucleic acid molecule B2 was the moiety that was reverse complementary to the 3′-end segment of the single-stranded nucleic acid molecule B1. The region underlined by the squiggle of the single-stranded nucleic acid molecule B2 was the transposon capture region.

Each of the molecular barcode 1, the molecular barcode 2 and the molecular barcode 3 consisted of ten nucleotides, where each nucleotide was any one of A, T, C and G. A total of 1536 types of molecular barcodes 1, 1536 types of molecular barcodes 2 and 1536 types of molecular barcodes 3 were disposed. Each magnetic bead contained multiple specific nucleic acid molecules that were the same (that is, all the specific nucleic acid molecules on each magnetic bead contained the same molecular barcode 1, the same molecular barcode 2 and the same molecular barcode 3). Hybridization capture sequence-contained magnetic bead carriers that contained the same specific nucleic acid molecule (that is, contained the same molecular barcode 1, the same molecular barcode 2 and the same molecular barcode 3) were considered as one type of hybridization capture sequence-contained magnetic bead carrier. For each hybridization capture sequence-contained magnetic bead carrier, other moieties of the specific nucleic acid molecules were the same except for sequences of the molecular barcode 1, the molecular barcode 2 and the molecular barcode 3. There were 1536×1536×1536 types of magnetic bead carriers in total.

4. Preparation of a mixed sample

The product solution of NA12878 obtained in step 2 and the product solution of the genomic DNA of Escherichia coli DH5α obtained in step 2 were taken and mixed in equal volumes to obtain a mixed sample 1.

The product solution of the genomic DNA of Escherichia coli DH5α obtained in step 2 and the product solution of the genomic DNA of Arabidopsis lyrata obtained in step 2 were taken and mixed in equal volumes to obtain a mixed sample 2.

The product solution of the genomic DNA of Escherichia coli DH5α obtained in step 2 and the product solution of Lambda DNA obtained in step 2 were taken and mixed in equal volumes to obtain a mixed sample 3.

The three mixed samples were placed on ice.

The three mixed samples obtained in step 4 were separately subjected to subsequent steps 5 to 10.

5. Capture of the barcoded DNA fragments

(1) The hybridization capture sequence-contained magnetic bead carrier prepared in step 3 was taken and added to a 1.5 ml centrifuge tube (magnetic beads were in an amount of 30×1.1 million), the centrifuge tube was placed on a magnet for 2 min until the liquid was clear, and the supernatant was discarded. The beads were washed with 1X low salt wash buffer (LSWB), and the supernatant was discarded. The beads were washed again with 1X LSWB, and the supernatant was discarded.

(2) After step (1) was completed, the centrifuge tube was added with 55 μl of capture buffer (containing 100 mM Tris-HCl with a pH of 7.5, 200 mM MgCl₂ and 0.1% Tween-20, and the balance was water) for resuspending.

(3) A new 1.5 ml centrifuge tube was taken and added with 50 μl suspension of the magnetic beads obtained in step (2) and 7.5 μl of a mixed sample obtained in step 4. The mixture was gently turned upside down ten times to be uniformly mixed, instantaneously centrifuged and incubated with rotation on a vertical mixer (incubated at 60° C. for 10 min and then at 45° C. for 50 min).

(4) After step (3) was completed, the centrifuge tube was taken and naturally cooled to room temperature, and added with 26 μl of ligation buffer I (containing 250 mM Tris-HCl with a pH of 7.5, 5 mM adenosine triphosphate (ATP) and 50 mM dithiothreitol (DTT), and the balance was water) and 4 μl of T4 DNA ligase (purchased from BGI, Cat. No. 01E004MM, with a concentration of 600 U/μl). The mixture was gently turned upside down ten times to be uniformly mixed, instantaneously centrifuged and incubated with rotation on a vertical mixer (incubated at 25° C. for 1 h).

6. Removing excess oligonucleotides on the magnetic beads through enzyme digestion

(1) After step 5 was completed, the centrifuge tube was taken and placed on a magnet for 2 min until the liquid was clear, and the supernatant was discarded. The beads were washed with 1X LSWB, and the supernatant was discarded.

(2) After step (1) was completed, the centrifuge tube was placed on ice and added with 95 μl of digestion buffer I (containing 33 mM Tris-HCl with a pH of 7.5, 66 mM potassium acetate, 10 mM magnesium acetate and 0.5 mM DTT, and the balance was water) and 5 μl of an exonuclease mixture (containing 3.75 μl of exonuclease I and 1.25 μl of exonuclease III). The mixture was gently turned upside down ten times to be uniformly mixed, instantaneously centrifuged and incubated on a vertical mixer (incubated at 37° C. for 10 min). Exonuclease I: purchased from BGI, Cat. No. 01E010ML, with a concentration of 20 U/μl. Exonuclease III: purchased from BGI, Cat. No. 01E011HL, with a concentration of 100 U/μl.

7. Release of the transposase through adding a denaturing agent

(1) After step 6 was completed, the centrifuge tube was added with 11 μl of 1% SDS aqueous solution, covered with a tube cap, shaken, uniformly mixed and incubated on a vertical mixer at room temperature for 10 min.

(2) After step (1) was completed, the centrifuge tube was instantaneously centrifuged and placed on a magnet for 2 min until the liquid was clear, and the supernatant was discarded.

(3) After step (2) was completed, the centrifuge tube was taken and washed three times. The steps of each washing were as follows: the centrifuge tube was added with 150 μl of 1X LSWB, shaken and placed on a magnet for 2 min until the liquid was clear, and the supernatant was discarded.

8. Addition of an adapter

(1) After step 7 was completed, the centrifuge tube was taken and added with 20 μl of pre ligation buffer (containing 50 mM Tris-HCl with a pH of 7.5 and 20 mM MgCl₂, and the balance was water) and 4 μl of pre ligation enzyme (single-strand DNA-binding (SSB) protein, purchased from BGI, Cat. No. BGE006, with a concentration of 500 μg/ml). The mixture was vortexed to be uniformly mixed and incubated on a vertical mixer at 37° C. for 30 min.

(2) After step (1) was completed, the centrifuge tube was taken and naturally cooled to room temperature, and added with 48 μl of ligation buffer II (containing 150 mM Tris-HCl with a pH of 7.8, 3 mM ATP, 1.5 mM DTT, 0.15 mM bovine serum albumin (BSA), 30 mM MgCl₂ and 30% PEG8000, and the balance was water), 18 μl of an adapter solution and 10 μl of T4 DNA ligase (purchased from BGI, Cat. No. 01E004MM, with a concentration of 600 U/μl). The mixture was vortexed to be uniformly mixed and incubated on a vertical mixer at room temperature for 2 h.

The active ingredient provided by the adapter solution was adapter. In the adapter solution, the adapter had a concentration of 16.67 μM. The adapter consisted of a single-stranded DNA molecule adapter-1A and a single-stranded DNA molecule adapter-2A.

Adapter-1A (Sequence 5):
5′phos-TCTGCTGAGTCGAGAACGTCT/3ddC/-3′.
Adapter-2A (Sequence 6):
5′-CTCGACTCAGCAG/3ddA/-3′.

“3ddC” refers to a cytosine dideoxyribonucleotide at the 3′-end, and “3ddA” refers to an adenine dideoxyribonucleotide at the 3′-end. 9. PCR amplification

(1) After step 8 was completed, the centrifuge tube was added with 80 μl of 1X LSWB and placed on a magnet for 2 min until the liquid was clear, and the supernatant was discarded.

(2) After step (1) was completed, the centrifuge tube was added with 180 μl of 1X LSWB and placed on a magnet for 2 min until the liquid was clear, and the supernatant was discarded.

(3) After step (2) was completed, the centrifuge tube was added with 2.25 μl of PCR enzyme and 147.75 μl of PCR buffer, uniformly mixed and subjected to the PCR amplification.

PCR enzyme: PfuTurbo Cx Hotstart DNA polymerase, purchased from Agilent Technologies, Inc., Cat. No. 600414, with a concentration of 2.5 U/μl.

PCR buffer contained 5% dimethylsulfoxide (DMSO), 1 M betaine, 6 mM MgSO₄, 0.6 mM deoxyribonucleoside triphosphate (dNTP), 0.5 μM PCR primer-F and 0.5 μM PCR primer-R.

PCR primer-F (Sequence 7):
5′-TGTGAGCCAAGGAGTTG-3′.
PCR primer-R (Sequence 8):
5′Phos-GAGACGTTCTCGACTCAGCAGA-3′.

Reaction parameters for the PCR amplification: hot cap function was performed at 105° C.; at 98° C. for 3 min; at 95° C. for 30s, at 58° C. for 30s, at 72° C. for 2 min, nine cycles; at 72° C. for 10 min; and held at 4° C.

(4) After step (3) was completed, the centrifuge tube was placed on a magnet for 2 min until the liquid was clear, and the supernatant was collected.

10. Purification of the PCR product

The supernatant obtained in step 9 was taken and purified using DNA clean beads to obtain a product solution (the solvent was TE buffer), that is, a library solution.

The library solution was taken and quantified using a Qubit™ double-stranded DNA high-sensitivity fluorescence quantification kit, and the DNA concentration was ≥3 ng/μL.

11. The library solution obtained in step 10 was taken and detected through electrophoresis.

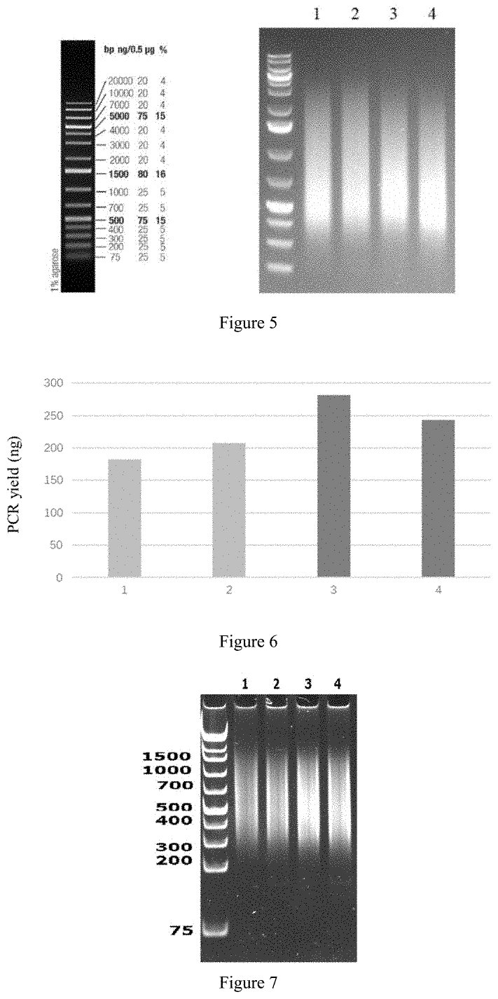

The results are shown in FIG. 5. In FIG. 5, Marker is GeneRuler 1 kb Plus DNA Ladder, the lane 1 corresponds to a library solution obtained from the mixed sample 1, the lane 2 corresponds to a library solution obtained from the mixed sample 2, and the lane 3 corresponds to a library solution obtained from the mixed sample 3.

### Example 3. An artificial sequence has higher interruption efficiency than a natural transposase recognition sequence

1. Preparation of a barcoded transposase complex C

(1) Preparation of a barcoded transposase-loading fragment

The barcoded transposase-loading fragment was formed of a single-stranded nucleic acid molecule A1 and a single-stranded nucleic acid molecule C (a natural transposase recognition sequence).

The barcoded transposase-loading fragment was prepared by a specific method as follows: the single-stranded nucleic acid molecule A1 and the single-stranded nucleic acid molecule C (both at a concentration of 100 NM) were mixed in equal volumes and subjected to annealing to obtain a product solution. Annealing parameters: at 70° C. for 3 min; cooled to 20° C. (with a cooling rate of 0.1° C./s), at 20° C. for 30 min, and held at 4° C. The product solution contained the barcoded transposase-loading fragment at a concentration of 50 μM.

Single-strandednucleic acid molecule
A1 (Sequence 1):
5′Phos-CGATCCTTGGTGATCNNNNNNNNNN [symbol] [symbol]
AGATGTGTATAAGAGACAG-3′.
Single-stranded nucleic acid molecule
C (Sequence 9):
5′Phos-CTGTCTCTTATACACATCT-3′.

In the single-stranded nucleic acid molecule A1, 10 N underlined by the straight line constituted a sample barcode, where N represented any one of A, T, C and G. Each sample corresponded to a unique sample barcode for distinguishing a source of the sample.

The bold moiety of the single-stranded nucleic acid molecule A1 and the single-stranded nucleic acid molecule C formed a double-stranded structure (the double-stranded structure was a transposase recognition region), and the remaining moiety was a single-stranded structure. The moiety underlined by the squiggle of the single-stranded nucleic acid molecule A1 was a spacer region, and the italic moiety of the single-stranded nucleic acid molecule A1 was a capture recognition region.

(2) Preparation of the barcoded transposase complex C

16.52 μl of Tn5 transposase (purchased from BGI, Cat. No. BGE005, with a concentration of 1 U/μl), 17.08 μl of coupling buffer (6.3+0.1 g glycerol dissolved in 5 ml TE buffer), 17.92 of μl TE buffer and 4.48 μl of the product solution obtained in step (1) were uniformly mixed on ice and incubated at 30° C. for 1 h to obtain a product solution C. The product solution C was stored at −20° C. until use. The product solution C contained the barcoded transposase complex C.

2. Preparation of a barcoded transposase complex A

(1) Preparation of a barcoded transposase-loading fragment

The barcoded transposase-loading fragment was formed of the single-stranded nucleic acid molecule A1 and a single-stranded nucleic acid molecule A2.

The barcoded transposase-loading fragment was prepared by a specific method as follows: the single-stranded nucleic acid molecule A1 and the single-stranded nucleic acid molecule A2 (both at a concentration of 100 NM) were mixed in equal volumes and subjected to annealing to obtain a product solution. Annealing parameters: at 70° C. for 3 min; cooled to 20° C. (with a cooling rate of 0.1° C./s), at 20° C. for 30 min, and held to 4° C. The product solution contained the barcoded transposase-loading fragment at a concentration of 50 μM.

Single-stranded nucleic acid molecule
A1 (Sequence 1):
5′Phos-CGATCCTTGGTGATCNNNNNNNNNN [symbol] [symbol]
AGATGTGTATAAGAGACAG-3′.
Single-stranded nucleic acid molecule
A2 (Sequence 2):
5′PhOS-CTGUCTCUTATACACAUCT-3′.

In the single-stranded nucleic acid molecule A1, 10 N underlined by the straight line constituted a sample barcode, where N represented any one of A, T, C and G. Each sample corresponded to a unique sample barcode for distinguishing a source of the sample.

The bold moiety of the single-stranded nucleic acid molecule A1 and the single-stranded nucleic acid molecule A2 formed a double-stranded structure (the double-stranded structure was a transposase recognition region), and the remaining moiety was a single-stranded structure. The moiety underlined by the squiggle of the single-stranded nucleic acid molecule A1 was a spacer region, and the italic moiety of the single-stranded nucleic acid molecule A1 was a capture recognition region.

(2) Preparation of the barcoded transposase complex A

16.52 μl of Tn5 transposase (purchased from BGI, Cat. No. BGE005, with a concentration of 1 U/μl), 17.08 μl of coupling buffer (6.3+0.1 g glycerol dissolved in 5 ml TE buffer), 17.92 μl of TE buffer and 4.48 μl of the product solution obtained in step (1) were uniformly mixed on ice and incubated for 1 h at 30° C. to obtain a product solution A. The product solution A was stored at −20° C. until use. The product solution A contained the barcoded transposase complex A.

3. Fragmentation and barcoding of high-molecular-weight DNA

The high-molecular-weight DNA was: NA12878 (CORIELL, Cat. No. NA12878).

10 ng of high-molecular-weight DNA was taken and added to a 0.2 ml centrifuge tube, and nuclease-free water was added to 38 μl. Then, 10 μl of 5×tagmentation buffer (purchased from BGI, Cat. No. BGE005B01) and 2 μl of 16-fold diluent (prepared by diluting the product solution C obtained in step 1 or the product solution A obtained in step 2 to 16-fold volume with TE buffer, which was performed on ice) were added, uniformly mixed and incubated at 55° C. for 10 min to obtain a product solution. The 0.2 ml centrifuge tube containing the product solution was transferred to ice. The product solution contained barcoded DNA fragments.

3. Release of the transposase through adding a denaturing agent

(1) After step 2 was completed, the centrifuge tube was added with 5 μl of 1% SDS aqueous solution, covered with a tube cap, shaken, uniformly mixed and incubated on a vertical mixer at room temperature for 10 min.

(2) After step (1) was completed, the centrifuge tube was instantaneously centrifuged and added with 67 μl of DNA clean beads for purification, and the mixture was dissolved in 20 μl of TE buffer.

4. Addition of an adapter

(1) After step 3 was completed, a new centrifuge tube was taken and added with 5 μl of product solution in step 3, 25 μl of ligation buffer II (containing 150 mM Tris-HCl with a pH of 7.8, 3 mM ATP, 1.5 mM DTT, 0.15 mM BSA, 30 mM MgCl₂ and 30% PEG8000, and the balance is water), 1.5 μl of an adapter solution, 1 μl of T4 DNA ligase (BGI, Cat. No. 01E004MM, with a concentration of 600 U/μl) and 18.5 μl of water. The mixture was vortexed to be uniformly mixed and incubated at room temperature for 1 h.

The active ingredient provided by the adapter solution was adapter. In the adapter solution, the adapter had a concentration of 16.67 μM. The adapter consisted of a single-stranded DNA molecule adapter-1A and a single-stranded DNA molecule adapter-2A.

Adapter-1A (Sequence 5):
5′phos-TCTGCTGAGTCGAGAACGTCT/3ddC/-3′.
Adapter-2A (Sequence 6):
5′-CTCGACTCAGCAG/3ddA/-3′.

“3ddC” refers to a cytosine dideoxyribonucleotide at the 3′-end, and “3ddA” refers to an adenine dideoxyribonucleotide at the 3′-end.

(2) After step (1) was completed, 60 μl of DNA clean beads were added for purification, and the mixture was dissolved in 20 μl of TE buffer. 5. PCR amplification

(1). The product solution in step 4 was added with 1 μl of PCR enzyme and 25 μl of PCR buffer 2, uniformly mixed and subjected to the PCR amplification.

PCR enzyme: PfuTurbo Cx Hotstart DNA polymerase, purchased from Agilent Technologies, Inc., Cat. No. 600414, with a concentration of 2.5 U/μl.

PCR buffer 2 contained 10% DMSO, 2 M betaine, 12 mM MgSO₄, 1.2 mM dNTP, 1 μM PCR primer 2-F and 1 μM PCR primer-R.

PCR primer 2-F (Sequence 10):
5′-TTGTCTTCCTAAGATGTGTATAAGAGACAG-3′.
PCR primer-R (Sequence 8):
5′-GAGACGTTCTCGACTCAGCAGA-3′.

Reaction parameters for the PCR amplification: hot cap function was performed at 105° C.; at 98° C. for 3 min; at 95° C. for 30s, at 58° C. for 30s, at 72° C. for 2 min, eleven cycles; at 72° C. for 10 min; and held at 4° C.

6. Purification of the PCR product

The product obtained in step 5 was taken and purified using DNA clean beads to obtain 20 μl product solution (the solvent was TE buffer).

The product solution in step 6 was taken and quantified using a Qubit™ double-stranded DNA high-sensitivity fluorescence quantification kit. A PCR yield was calculated after the quantification. The results are shown in FIG. 6. In FIG. 6, 1 and 2 correspond to the product solution C obtained in step 1 (two repetitions, respectively), and 3 and 4 correspond to the product solution A obtained in step 2 (two repetitions, respectively).

The product solution in step 6 was taken and detected through electrophoresis. The results are shown in FIG. 7. In FIG. 7, Marker is GeneRuler 1 kb Plus DNA Ladder, lanes 1 and 2 correspond to the product solution C obtained in step 1 (two repetitions, respectively), and lanes 3 and 4 correspond to the product solution A obtained in step 2 (two repetitions, respectively).

### INDUSTRIAL APPLICATION

The present disclosure has the following functions: (1) the present disclosure provides a stLFR-based multi-sample mixed library construction technology, which successfully solves the problems of mixed library construction and sequencing of large samples; (2) the present disclosure may significantly reduce the complexity of library construction, improve throughput of the library construction, improve a utilization rate of a sequencing instrument and reduce costs of library construction and sequencing for a single sample; (3) the present disclosure is applicable to resequencing and de novo assembly of samples with a small genome and samples with a requirement for a specific amount of data; (4) the present disclosure may further reduce an initial starting amount of a single sample to less than 1.5 ng, which is applicable to resequencing and de novo assembly of rare samples and samples in very low biomass; and (5) high-throughput automated library construction is convenient to be achieved.

1. A transposase recognition element, which is characterized by the following (a) and/or (b):
(a) a transferred strand contains a fixed sequence;
(b) a non-transferred strand contains a U base.

2. A transposase recognition element, which is characterized in that:
the transposase recognition element has a structure of X(m)Y(f)N(n); wherein
X(m) denotes a transposase recognition region and has a double-stranded nucleic acid structure;
Y(f) denotes a spacer region and has a single-stranded DNA structure;
N(n) denotes a sample barcode and has a single-stranded DNA structure;
optionally, in the transposase recognition region, a portion of T in one strand is replaced with U.

3. (canceled)

4. A barcoded transposase complex, which is formed of a transposase and a transposase recognition element;
wherein the transposase recognition element is the transposase recognition element according to claim 1.

5. A method for preparing a barcoded DNA fragment, comprising the following steps: providing high-molecular-weight DNA and treating with the barcoded transposase complex according to claim 4.

6. A method for constructing a DNA library, comprising the following steps in sequence:
(1) providing high-molecular-weight DNA and preparing a barcoded DNA fragment using the method according to claim 5; and
(2) treating with an exonuclease and releasing the transposase.

7. A method for constructing a DNA library, comprising the following steps in sequence:
(1) providing high-molecular-weight DNA and preparing a barcoded DNA fragment using the method according to claim 5;
(2) capturing with a carrier containing a molecular barcode; and
(3) treating with an exonuclease and releasing the transposase.

8. A method for constructing a DNA library, comprising the following steps in sequence:
(1) providing n pieces of high-molecular-weight DNA and preparing barcoded DNA fragments using the method according to claim 5, respectively, wherein n is a natural number greater than or equal to 2;
(2) mixing the barcoded DNA fragments obtained after each high-molecular-weight DNA is subjected to step (1), to obtain a mixed sample; and
(3) treating with an exonuclease and releasing the transposase;
optionally, the method further comprises the following step:
(4) performing library construction using a single-tube long fragment read (stLFR) technology to obtain the DNA library.

9. (canceled)

10. A method for constructing a DNA library, comprising the following steps in sequence:
(1) providing n pieces of high-molecular-weight DNA and preparing barcoded DNA fragments using the method according to claim 5, respectively, wherein n is a natural number greater than or equal to 2;
(2) mixing the barcoded DNA fragments obtained after each high-molecular-weight DNA is subjected to step (1), to obtain a mixed sample;
(3) capturing the mixed sample obtained in step (2) with a carrier containing a molecular barcode; and
(4) treating with an exonuclease and releasing the transposase;
optionally, the method further comprises the following step:
(5) performing library construction using a single-tube long fragment read (stLFR) technology to obtain the DNA library.

11. (canceled)

12. A kit for preparing a barcoded DNA fragment, comprising a transposase and a transposase recognition element, wherein the transposase recognition element is the transposase recognition element according to claim 1.

13. (canceled)

14. A kit for constructing a DNA library, comprising a transposase and a transposase recognition element, wherein the transposase recognition element is the transposase recognition element according to claim 1.

15. (canceled)

16. Use of the transposase recognition element according to claim 1 in DNA sequencing.

17. (canceled)

18. (canceled)

19. (canceled)

20. A barcoded transposase complex, which is formed of a transposase and a transposase recognition element;
wherein the transposase recognition element is the transposase recognition element according to claim 2.

21. A method for preparing a barcoded DNA fragment, comprising the following steps: providing high-molecular-weight DNA and treating with the barcoded transposase complex according to claim 20.

22. A method for constructing a DNA library, comprising the following steps in sequence:
(1) providing high-molecular-weight DNA and preparing a barcoded DNA fragment using the method according to claim 21; and
(2) treating with an exonuclease and releasing the transposase.

23. A method for constructing a DNA library, comprising the following steps in sequence:
(1) providing high-molecular-weight DNA and preparing a barcoded DNA fragment using the method according to claim 21;
(2) capturing with a carrier containing a molecular barcode; and
(3) treating with an exonuclease and releasing the transposase.

24. A method for constructing a DNA library, comprising the following steps in sequence:
(1) providing n pieces of high-molecular-weight DNA and preparing barcoded DNA fragments using the method according to claim 21, respectively, wherein n is a natural number greater than or equal to 2;
(2) mixing the barcoded DNA fragments obtained after each high-molecular-weight DNA is subjected to step (1), to obtain a mixed sample; and
(3) treating with an exonuclease and releasing the transposase;
optionally, the method further comprises the following step:
(4) performing library construction using a single-tube long fragment read (stLFR) technology to obtain the DNA library.

25. A method for constructing a DNA library, comprising the following steps in sequence:
(1) providing n pieces of high-molecular-weight DNA and preparing barcoded DNA fragments using the method according to claim 21, respectively, wherein n is a natural number greater than or equal to 2;
(2) mixing the barcoded DNA fragments obtained after each high-molecular-weight DNA is subjected to step (1), to obtain a mixed sample;
(3) capturing the mixed sample obtained in step (2) with a carrier containing a molecular barcode; and
(4) treating with an exonuclease and releasing the transposase;
optionally, the method further comprises the following step:
(5) performing library construction using a single-tube long fragment read (stLFR) technology to obtain the DNA library.

26. A kit for preparing a barcoded DNA fragment, comprising a transposase and a transposase recognition element, wherein the transposase recognition element is the transposase recognition element according to claim 2.

27. A kit for constructing a DNA library, comprising a transposase and a transposase recognition element, wherein the transposase recognition element is the transposase recognition element according to claim 2.

28. Use of the transposase recognition element according to claim 2 in DNA sequencing.
